(19) United States
(12) Patent Application Publication
(10) Pub. No.: US 2004/0260257 A1
(43) Pub. Date: Dec. 23, 2004

(54) Ostomy sealing device

(76) Inventors: Danuta Ciok, Nivaa (DK); Dorrit Diana Israelson, Gentofte (DK)

Correspondence Address: JACOBSON HOLMAN PLLC, 400 SEVENTH STREET N.W., SUITE 600, WASHINGTON, DC, 20004, US

(21) Appl. No.: 10/491,126

(22) PCT Filed: Sep. 25, 2002
(86) PCT No.: PCT/DK02/00631

(30) Foreign Application Priority Data
Sep. 28, 2001 (DK) PA 2001 01428

(51) Int. Cl. A61F 5/44

(52) U.S. Cl. 604/332

(57) ABSTRACT

A disposable closure for an artificial intestinal or urethral opening in the form of a body having a cup form having an internal space being capable of accommodating a stoma, characterised in that the rim of the open end of the cup is provided with a flange part stretching essentially perpendicularly to the longitudinal direction of the closure and that the surface of the flange facing away from the closed end of the cup is provided with a skin-friendly adhesive may be used for safe temporary closing of a stoma without the risk of causing mechanical damage to the stomal wall and which prevents intestinal contents from passing the closure and which is easy to remove after use. It may also be used for protection of the outer surface of a stoma when applying a fresh collecting bag and furthermore as an aid for the user for an easier manner to place a fresh body side member or a one-piece receiving bag “correctly” ensuring a correct coupling and sealing.

### BACKGROUND OF THE INVENTION

1. Field of the Invention

The present invention relates to a disposable closure for an artificial intestinal or urethral opening in the form of a body having a cup form having an internal space being capable of accommodating a stoma.

In connection with surgery for a number of diseases in the gastrointestinal tract a consequence is, in many cases, that the patient is left with a stoma such as a colostomy, an ileostomy or an urostomy in the abdominal wall for the discharge of the effluents or waste products of the body, which are conveyed through the colon, the ileum or the ureter. The discharge of visceral contents including intestinal gases cannot be regulated at will, and for that purpose the opening may be closed with a closure means, e.g. a tampon or a magnetic closure, or the patient will have to rely on an appliance to collect the material emerging from such opening in the form of a receiving bag which is later emptied and/or discarded at suitable times.

Ostomy appliances are well known. Such appliances may be two-piece or one-piece appliances. In both types of appliances, an adhesive wafer is attached to the wearer's skin. In case of a one-piece appliance, a receiving member or bag is attached to the adhesive wafer. In case of a two-piece appliance, the adhesive wafer forms part of a body side member and a receiving bag is attached releasably to the body side ostomy member for receiving exudates from the stoma.

When using one-piece appliances, the whole appliance, including the adhesive skin barrier securing the appliance to the skin is normally removed and replaced by a fresh appliance. When using two-piece appliances, the body side member is left in place up to several days, and only the receiving bag attached to the body side member is replaced.

2. Description of the Related Art

Most often, the visceral contents from a stoma are collected in bags but frequently problems occur with respect to contamination of e.g. a body side member of a two-piece appliance when substituting the collecting bag with a fresh, or the peristomal skin may be contaminated with the aggressive secretions from the stoma when substituting a one-piece appliance before applying a fresh appliance which may lead to improper adhesion to the skin and leaks.

Furthermore, faeces from an incontinent natural anal opening are sometimes collected by means of diaper-like appliances and also in this connection problems are encountered when substituting a used appliance with a fresh. DE-A-2 363 563 and its addition DE-A-2 447 682 as well as corresponding other specifications, e.g. GB-A-1 471 158, propose to close artificial intestinal openings with a magnetic plug held by a ring magnet surgically implanted around the portion of the intestine adjacent the surface of the body. A seal against unintended discharge of intestinal contents is provided between a plate shaped part of the plug and the skin, which necessitates a rather strong magnetic action which in many cases is uncomfortable and in adverse cases may cause some tissue necrotization. Closures of this type are not suitable for very fat patients, for patients having varying weight and for patients in which the outer part of the intestine is oblique relative the skin surface, because in these cases there are big difficulties in rendering the closure fluid-tight. Another type of closure has therefore been developed, namely a closure plug of a suitable soft, and possibly weakly elastic, material to be inserted into the part of the intestinal duct in question adjacent to the body surface. An example of such a closure is known from DE-A-2 717 608 and consists of a magnet or magnetic core surrounded by a tampon-like material which is expandable on insertion into the intestinal duct or the anus, the closure being meant for both an incontinent natural anus and for ostomies.

As the expandable material, there are proposed materials which expand under the influence of heat or moisture, e.g. cellulosic materials of largely the same kinds as those used in catamenial tampons. The core of magnetic material or the magnet co-operates with a ring magnet implanted in the tissue surrounding the outer parts of the intestine. An analogous closure without magnetic holding means is described in U.S. Pat. No. 4,209,009 according to which a closing tampon for an incontinent natural or an artificial intestine opening consists of an elongate substantially cylindrical body of a cellular material being non homogenous in the longitudinal direction. This body is formed of longitudinally aligned parts having different radial expansion properties when wetted, first and a third sections having high expansion characteristics when wetted and being separated by a second intermediate section having lesser expansion characteristics when wetted. A holding ring of non-expanding material is surgically implanted around the intestinal opening, and in use the second section of the tampon is situated within this ring.

A further example of this type of closure is described in DE-A-2 717 607. It has similar magnetic holding means as the closure according to DE-A-2 717 608 but the magnetic core is surrounded by a soft elastic, radially compressible material such as a foam plastic and that material is the proper closure means. None of these more or less tampon-like arrangements has achieved a broad acceptance. This is presumed to be due to the fact that the proper principle of closing depends upon the absorption of liquid into cellulosic material or foam material being of fundamentally the same kind as is used in catamenial tampons, and that the absorption of liquid in these is not always sufficiently rapid as to avoid leakage in the time immediately after the insertion. The pressure against the intestinal wall is low and the sealing consequently often unsatisfactory. In cases where the tampons have so large a diameter before insertion that the seal is actually obtained because of the shape of the tampon, insertion as a rule will be difficult because compression has to take place, and this may be accompanied by discomfort or pain and risk of damaging the intestinal wall because the surface of the tampon may not be smooth.

A closure in which the sealing effect relies upon absorption of liquid into an essentially inelastic material is not very suitable for intestinal use where the pressure behind it, caused for example by intestinal gas, will tend to expel the closure or allow not only intestinal gas but also other contents of the intestine to bypass the closure between the sealing and the intestinal wall.

EP patent No. 188 376 owned by applicant discloses a disposable closure for an intestinal opening comprising an elastic body which is held in a compressed state, prior to insertion, by a material which is sensitive to heat and/or moisture such that it ceases to hold the body in the compressed state after insertion of the closure into the intestinal opening so that the body expands under the effect of its elasticity to seal the opening. The closure disclosed in EP 188 376 is insoluble and may be provided with a string for use in removal from the intestine.

The closures of the art do not offer a solution to the problems encountered when it is desired to have a temporary closure of the stoma while substituting a used appliance with a fresh as they must be pulled out of the stoma after use which will be impossible or at least rather troublesome after applying a fresh collection bag.

One solution to this problem is proposed in WO 00/47143 in the form of a disposable closure made from a material being soluble in visceral contents. This solution seals the stoma ensuring that visceral contents or urine does not ooze from the stoma and also provides time for cleaning the stomal area before applying a fresh collecting bag and for preparing the fresh bag, e.g. by adapting the size of the inlet hole thereof.

WO 00/47143 also discloses a disposable closure made from a material being soluble in visceral contents in the form of a cup shaped closure having a narrow waist part for engaging with the outer surface of the stoma. Such cup shaped closure may hurt or even harm the stoma if a cup of a too small opening is. The risk of such problems is not negligible as the cup must be narrow enough to engage with the outer surface of the stoma for ensuring a safe sealing.

Even this solution, however, requires the user to push the closure into the stoma or to place it in engagement with the outer surface of the stoma which may be difficult and furthermore, there may still be a problem for many users in aligning the fresh collecting bag with the stoma and placing the bag on the abdomen or body side member, especially if a coupling system utilising matching flanges for adhesive connection is used.

U.S. Pat. No. 3,954,105 discloses a combination of a sheet of a thin pliable material adapted to be placed in sealing engagement with the skin of a patient in the location of a body opening made by an incision or wound, with the sheet being adapted to have an aperture formed therein to expose the opening, together with a removable cap which can be adhesively secured to the outer surface of the sheet. However, this disclosure does not, either, offer a solution the problems encountered when it is desired to have a temporary closure of the stoma while substituting a used appliance and also to the problems associated with the placing of a fresh ostomy body side member of a one-piece ostomy appliance.

It is therefore desirable to provide a disposable closure for an intestinal opening that does not have the drawbacks of the known closures and which may be used for temporary closing of a stoma without the risk of causing mechanical damage to the stomal wall and which prevents intestinal contents from passing the closure and which is easy to remove after use. There is also a need for a protection of the outer surface of a stoma when cleaning a body side member before applying a fresh collecting bag and furthermore for an aid for the user for an easier manner to place a fresh body side member or a one-piece receiving bag “correctly” ensuring a secure positioning and sealing against the skin.

### SUMMARY OF THE INVENTION

The present invention relates to a disposable closure for an artificial intestinal or urethral opening in the form of a body having a cup form having an internal space being capable of accommodating a stoma.

### BRIEF DESCRIPTION OF THE DRAWINGS

The invention is disclosed more in detail with reference to the drawings in which

FIG. 1 shows an embodiment of a closure device of the invention seen from the open end,

FIG. 2 shows a sectional view of the closure device shown in FIG. 1,

FIG. 3 shows the use of a closure of the invention for protecting a stoma during cleaning of a body side member, and

FIG. 4 shows the use of a closure of the invention for protecting a stoma during application of a body side member.

### DETAILED DESCRIPTION OF THE PRESENT INVENTION

The invention relates to a disposable closure for an artificial intestinal or urethral opening in the form of a body having a cup form having an internal space being capable of accommodating a stoma, said closure being characterised in that the rim of the open end of the cup is provided with a flange part stretching essentially perpendicularly to the longitudinal direction of the closure and that the surface of the flange facing away from the closed end of the cup is provided with a skin-friendly adhesive.

A closure of the invention may be used for safe temporary closing of a stoma without the risk of causing mechanical damage to the stomal wall when cleaning a body side member before applying a fresh collecting bag. In this case, the diameter of the closure ensures that the closure fully accommodates the stoma and the flange seals against the top surface of the ostomy body side member. The closure of the invention furthermore may serve as an aid for the user for an easier manner to place a fresh body side member or a one-piece receiving bag “correctly” ensuring a correct placing and sealing against the skin. In this case, the closure fully accommodates the stoma and the flange seals against the surface of the skin surrounding the stoma. When used for correct placing, the flange part should be sufficiently thin and flexible to allow an easy removal from below the adhesive wafer. The adhesive forces should preferably be balanced in consideration of this aspect and will easily be determined by routine experiments by the skilled in the art.

It is preferred that the height and width are of sufficient magnitudes to leave an internal space capable of fully accommodating the stoma and allow the flange to be in contact with the skin surrounding the basis of stoma along its full perimeter. This ensures that the flange will seal against a body side member or the skin next to the stoma and will provide a seal preventing material exiting from the stoma to spread over the area next to the stoma. Thus, it is preferred that the closure has a diameter only slightly greater than the diameter of the stoma with which it is intended to be used. Furthermore, it is preferred that the outer diameter of the flange is as small as possible without compromising a secure fastening and sealing leaving as large an open area as possible inside the coupling means free for cleaning.

It has been found suitable when the body of the closure is made from a stable and relative soft material. Such a material will be sufficiently self-supporting to be stable so that it may easily pass over and accommodate the stoma and at the same time the closure is not so stiff as to increase the risk of mechanical damaging of the stoma when applying or removing the closure.

It has been found especially suitable when the body is made from a silicone rubber material which is easily imparted the properties stated above and which is skin friendly.

The use of a skin-friendly adhesive is preferred as it is supposed to be in contact with the sensible skin next to a stoma. The adhesive should preferably have a sufficient adhesion to ensure a safe grip of the closure to the skin and, at the same time preferably be sufficiently easy to remove so as to allow a manipulation of the closure to release the grip using one or more finger(s) pushing or seizing the closure through the wall of a receiving bag without any danger of damaging the stoma, a base plate or the wall of the receiving bag which might lead to leakage or reduced service time for the appliance.

A very suitable and skin-friendly adhesive which may be used for the present invention is made from a permanently tacky silicone adhesive as such material is relatively non-sensible in connection with cleaning using water and does not bind materials or odour emerging from a stoma.

The closure is preferably made from a silicone material as silicone materials are generally skin-friendly and very stable when contacted by aggressive exudates from a stoma. Furthermore, silicones are often transparent enabling an inspection of the stoma. A preferred material for the closure is a silicone rubber being relatively inert for aggressive fluids and being washable using water.

Suitable adhesive materials are platinum catalysed vinyl endblocked polydior-ganosiloxanes of the kind described in U.S. Pat. No. 3,983,298, which have high tack, good adhesive strength. The properties and tack of these polysiloxanes may be adjusted according to wish by routine experiments by the skilled in the art for preparation of more tacky materials for adhering to the skin than if e.g. the surface of the flange which is to be adhered to an ostomy body side member. A preferred material is vinyl endblocked crosslinked polydimethylsiloxanes. Furthermore, it is preferred that the surfaces of the closure facing away from the user is hydrophobic as this facilitates the cleaning thereof.

The closure of the invention may be elastic enabling a temporary enlargement of the hole for receiving the stoma whereafter it reverts to substantially its original size as long as the elastic force is not so strong that there is a risk of constricting the stoma. However, it is preferred that the space inside the closure has dimensions exceeding the corresponding dimensions of the stoma enabling a safe accommodation and protection of the stoma and a safe sealing against the ostomy body side member or the skin.

Another very suitable and skin-friendly adhesive which may be used for the present invention is a hydrogel adhesive.

The closure of the invention is suitable for use in connection with conventional two-piece appliances and one-piece appliances.

### DESCRIPTION OF THE PREFERRED EMBODIMENTS

The invention is now explained more in detail with reference to the drawings showing preferred embodiments of the invention.

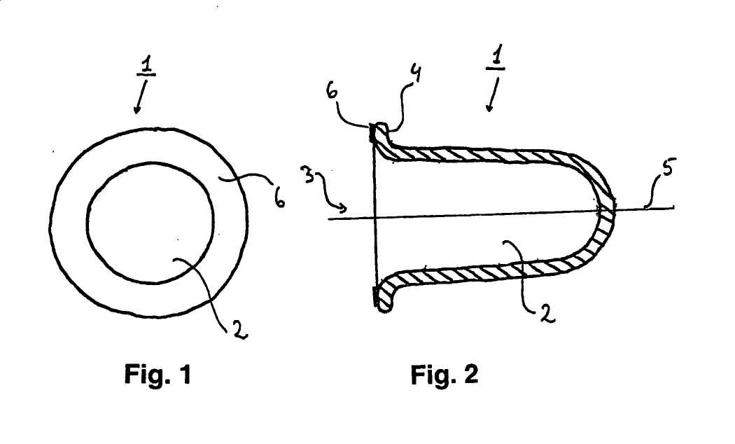

Reference is made to FIGS. 1 and 2 which show an embodiment of a closure device generally designated 1 of the invention in the form of a body having a cup form having an internal space 2 being capable of accommodating a stoma seen from the open 3 end and in the form of a sectional view. The open end of the cup is provided with a flange part 4 stretching essentially perpendicularly to the longitudinal direction indicated by a line 5 and the surface of the flange facing away from the closed end of the cup is provided with a skin-friendly adhesive 6.

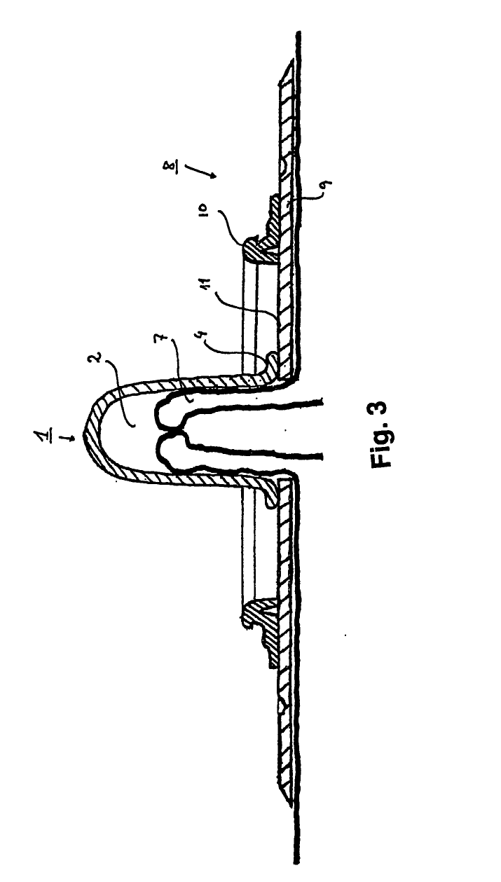

FIG. 3 shows the use of a closure 1 of the invention for protecting a stoma 7 during cleaning of a body side member 8 comprising an adhesive wafer 9 for adhering to the user's skin and a coupling ring 10 for attaching a receiving bag (not shown). The closure fully accommodates the stoma and the flange 4 seals against the top surface 11 of the ostomy body side member. Thus, it is possible to clean the top surface of the ostomy body side member and the coupling ring, even using mild detergents, without risk of irritating surface of the stoma which is often very sensitive as it is normally formed by a mucosal surface. After cleaning, the closure 1 may be removed before or after applying a fresh receiving bag as desired. In case of an ileostomy or an urostomy it may often be suitable to manipulate the closure through the wall of the receiving bag after applying the same as there will most probably be a minor amount of visceral contents of urine in the space 2 of the closure above the stoma which material might soil the coupling area 10 before the fresh receiving bag is in place if the closure is removed in advance.

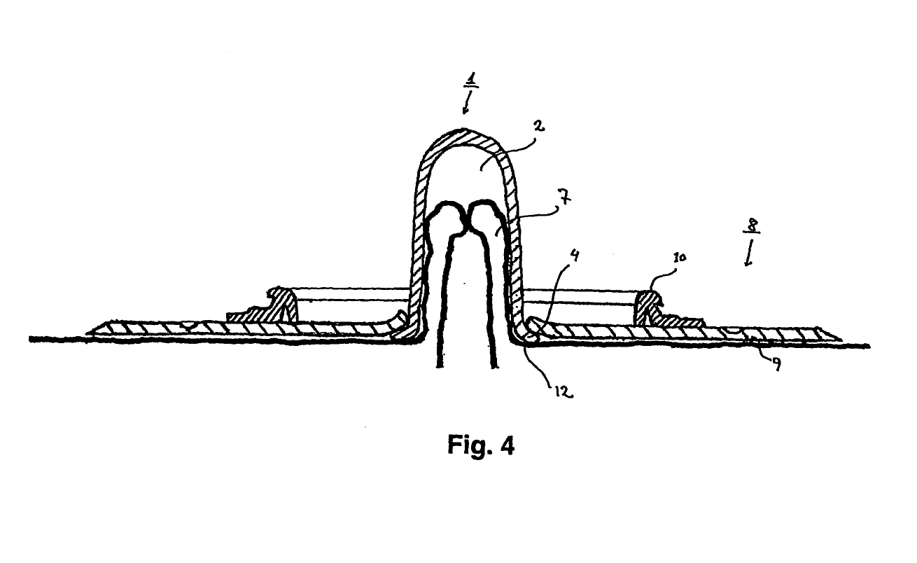

FIG. 4 shows the use of a closure 1 of the invention for protecting a stoma 7 during application of a body side member 8. In this case, the closure 1 fully accommodates the stoma 7 and the flange 4 seals against the surface of the skin 12. Thus, the closure partly protects the stoma from mechanical damage and partly functions as a guide for guiding the body side member along the stoma and to engage with the skin next to the stoma.

1. A disposable closure for an artificial intestinal or urethral opening in the form of a unitary body having a cup form having an internal space being capable of accommodating a stoma, said stoma projecting from the surface of the skin of the user, in combination with an ostomy body side member or a one-piece receiving bag, characterised in that the rim of the open end of the body is provided with a flange part stretching essentially perpendicularly to the longitudinal direction of the closure and that the surface of the flange facing away from the closed end of the body is provided with a skin-friendly adhesive, wherein the closure has a diameter only slightly greater than the diameter of the stoma with which it is intended to be used and the outer diameter of the body is sufficiently small to pass through the hole of the ostomy body side member or one-piece receiving bag for accommodating the stoma, and the outer diameter of the flange is so small so as to leave an area inside the coupling means free.

2. A closure as claimed in claim 1, characterised in that the height and width are of sufficient magnitudes to leave an internal space capable of fully accommodating the stoma and allow the flange to be in contact with the skin surrounding the basis of stoma along its full perimeter.

3. A closure as claimed in claim 1, characterised in that the body of the closure is made from an initially stable and relative soft material.

4. A closure as claimed in claim 3, characterised in that the body is made from a silicone rubber material.

5. A closure as claimed in claim 1, characterised in that the adhesive is a silicone adhesive.

6. A closure as claimed in claim 1, characterised in that the adhesive is a hydrogel adhesive.
